(19) United States
(12) Patent Application Publication
(10) Pub. No.: US 2020/0383983 A1
(43) Pub. Date: Dec. 10, 2020

(54) CGRP ANTAGONISTS FOR THE TREATMENT OF MEDICATION OVERUSE HEADACHE, POST-TRAUMATIC HEADACHE, POST-CONCUSSION SYNDROME AND VERTIGO

(71) Applicant: Allergan Sales, LLC, Madison, NJ (US)

(72) Inventors: Mitchell F. Brin, Newport Beach, CA (US); Andrew M. Blumenfeld, Del Mar, CA (US)

(21) Appl. No.: 16/852,173

(22) Filed: Apr. 17, 2020

Related U.S. Application Data
(60) Provisional application No. 62/835,591, filed on Apr. 18, 2019.

(51) Int. Cl. A61K 31/506; A61K 31/4196; A61K 31/4045; A61K 31/454; A61K 31/497; A61K 31/422; C07K 16/28

(52) U.S. Cl. CPC A61K 31/506; A61K 31/4196; A61K 31/4045; A61K 45/06; A61K 31/497; A61K 31/422; C07K 16/2869; A61K 31/454

(57) ABSTRACT

The application provides methods for treating, preventing, alleviating or reducing the frequency of occurrence of medication overuse headache, post-traumatic headache, post-concussion syndrome and vertigo in patients by the administration of calcitonin gene-related peptide (CGRP-antagonists). The CGRP-antagonists can be selected from ubrogepant or atogepant, or a pharmaceutically acceptable salt, ester or prodrug thereof. Anti-CGRP antibodies, such galcanezumab, fremanezumab, eptinezumab, and erenumab can also be used.

### FIELD

The application is related to medicaments and methods for treating medication overuse headache (MOH), post-traumatic headache, post-concussion syndrome and vertigo. The application further relates to the use of calcitonin gene-related peptide (CGRP-antagonists) for the treatment of MOH, post-traumatic headache, post-concussion syndrome and vertigo.

### BACKGROUND

Daily or near-daily headache can affect up to 5% of some populations, and it is believed that chronic overuse of headache drugs may account for half of this phenomenon. All simple analgesics, and probably non-steroidal anti-inflammatory drugs, ergotamine, and triptans, are implicated. Medication overuse headache affects more women than men (5:1) and some children.

A significant portion of the those suffering from headache can be classified as having posttraumatic headache. Approximately 2% of the US population is disabled secondary to posttraumatic headache. (Channell, M. K. et. al., JAOA, 109 (9) September 2009, 511). Posttraumatic headache, has been defined in several reports, including the International Classification of Headache Disorders, 2nd edition (ICHDII), the American Congress of Rehabilitation Medicine (ACRM), and the International Classification of Diseases, 10^{th} edition (ICD-10). Mild posttraumatic headache, when acute, lasts less than 3 months and is a trauma-induced physiologic disruption of brain function, as manifested by at least one of several symptoms. The multiple biochemical effects resulting from physical trauma in patients with posttraumatic headache are thought to lead to the pain and cognitive effects of head trauma. (Channell, M. K. et. al.)

The regular intake of three or more analgesic tablets daily or narcotics or ergotamine on more than two days a week to control or alleviate a headache has been suggested as a medication overuse headache definition. A common and probably important factor in medication overuse headache is pre-emptive use of drugs, in anticipation of rather than for a headache. Medication overuse headache usually does not develop when analgesics are regularly taken for another indication, such as chronic backache or rheumatic disease, that is the headache must be present to begin with.

A presumptive diagnosis of medication overuse headache is based on symptoms and a detailed history of drug use, including over the counter drugs. Many patients with medication overuse headache disorder use large quantities of drug: 35 doses a week on average in one study, and six different agents. Sooner or later, such patients seek prescriptions for “something stronger,” bringing them to the general practitioner's attention. However, medication overuse headache is typically confirmed only when symptoms improve after drugs are withdrawn. The headache is oppressive, present, and often at its worst on awakening in the morning. It can be increased after physical exertion. Associated nausea and vomiting are rarely pronounced. A typical history begins with episodic headache up to years earlier (more commonly migraine than tension-type headache), treated with an analgesic or other acute medication. Over time, headache episodes become more frequent, as does drug intake, until both are daily. In the end stage, which not all patients reach, headache persists all day, fluctuating with medication use repeated every few hours. This evolution occurs over a few weeks or much longer, depending largely but not solely on the medication taken.

The International Headache Society defines medication overuse headache (MOH) as a chronic headache (headache frequency>15 days per month) after the intake of analgesics or ergots (more than 15 times per month for at least 3 months), which disappears after withdrawal therapy. It has been described as a self-sustaining, rhythmic, headache medication cycle characterized by daily or near daily headache and irresistible and predictable use of immediate relief medications. Evidence supporting the existence of MOH is widely published in the medical literature.

The pathogenesis of MOH has not been fully elucidated. Some evidence suggests that up regulation of serotonin receptors and subsequent reduction in serotonin levels, which normalize upon cessation of chronic analgesic use, may play a role. The following have also been implicated in the development of MOH: endorphin suppression, central opioid receptor impairment, impaired suppression or downregulation of an already partly suppressed or abnormal antinociceptive system, alterations in density and function of postsynaptic neuronal receptors, and activation of nociceptive “on-cells” in the ventral medulla that facilitate nociceptive reflex responses. A common presentation is a patient with a history of episodic migraine with or without aura, who complains of increased headache frequency and the development of interparoxysmal tension-type headache, that eventually transforms into a daily or near-daily headache lasting for prolonged periods. Patients may alternate between migraine-type and tension-type headaches during this period. Behavioral and psychiatric comorbidities may also be present and are complicating factors. It is common for patients to underestimate their use of analgesics and to use multiple types of agents concomitantly. Initially, pain relief provides negative reinforcement, and in some cases changes in mood incurred from barbiturate and caffeine-containing analgesics, may provide positive reinforcement, resulting in excessive use. Tolerance, characterized by increasing consumption without regard to potential adverse outcomes, and withdrawal symptoms upon abrupt discontinuation, often ensue and result in increased headache frequency and severity with a decrease in analgesic efficacy. Concomitant preventive medications are relatively ineffective, while the patient is using excessive amounts of abortive agents and complete discontinuation of headache medication is the treatment of choice. Detoxification is usually conducted slowly over as many as 8 to 12 weeks and in the most severe cases, may warrant hospitalization.

MOH has been recently recognized as a unique disorder diagnosis of the International Classification of Headache Disorders, 2.sup.nd edition, published in supplement 1, Cephalalgia 2004: volume 24, pages 94-95, wherein, significantly, it is stated that patients with a MOH disorder rarely respond to other preventive medications while they are overusing their acute pain medications.

While the various types of headaches are widespread, the specific types of headaches with specific causal links, including MOH and post-traumatic headache need medications suitable to address their unique challenges.

### SUMMARY

The application provides methods for treating, preventing, alleviating or reducing the frequency of occurrence of medication overuse headache (MOH) in patients by the administration of calcitonin gene-related peptide (CGRP-antagonists). The application further provides methods for treating, preventing, alleviating or reducing the frequency of occurrence of post-traumatic headache (PTH), post-concussion syndrome and vertigo by the administration of CGRP-antagonists.

In some embodiments, the CGRP-antagonist is ubrogepant, atogepant, or a pharmaceutically acceptable salt, ester or prodrug thereof. In some embodiments, the CGRP-antagonist is an antibody selected from galcanezumab, fremanezumab, eptinezumab, and erenumab.

### DESCRIPTION

Provided are methods for treating, preventing, alleviating or reducing the frequency of occurrence of medication overuse headache (MOH) in patients by the administration of calcitonin gene-related peptide (CGRP-antagonists). In some embodiments, the application provides methods for the treatment of MOH induced by analgesics, non-steroidal anti-inflammatory drugs, ergotamine, and/or triptans by the administration of CGRP-antagonists. In some embodiments, the CGRP-antagonist is ubrogepant, atogepant, or a pharmaceutically acceptable salt, ester or prodrug thereof. In some embodiments, the CGRP-antagonist is an antibody or a fragment thereof. For example, an anti-CGRP antibody can be selected from galcanezumab, fremanezumab, eptinezumab, and erenumab. The methods herein provide significant reduction in the frequency, duration, or severity of headaches and related symptoms for patients suffering from MOH.

Without being bound by any particular theory, it is postulated that CGRP is an important component of the neurochemical cascade of headache. It is a potent vasodilator, involved in the production of inflammation and activation of meningeal nociceptors. The trigeminal ganglion involves neurons which are bipolar with the distal synapse innervating the meninges and the blood vessels in the meninges; and the proximal synapse projecting onto second order neurons in the Trigeminal Nucleus Caudalis (TNC). One of the presumed mechanisms of headache is that the Trigeminal Ganglion becomes activated. This activation leads to peripheral sensitization with dilation and inflammation of meningeal blood vessels; and central sensitization due to activation of the TNC. CGRP is released at both the distal and proximal synapses and thus is involved with both peripheral and central sensitization.

CGRP predominantly binds to the calcitonin receptor-like receptor (CLR) and receptor activity-modifying protein 1 (RAMP1) complex. However, CGRP is capable of activating multiple receptors and could have more than one endogenous receptor. One of these is the CGRP-responsive calcitonin receptor/RAMP1 complex (AMY₁ receptor-amylin subtype 1 receptor) in the trigeminovascular system. There are two types of Monoclonal antibodies directed against CGRP, those that bind the CLR and RAMP1 receptors (Erenumab) and those that bind the CGRP Ligand (Galcanezumab, Fremanezumab, and Eptinezumab). The CGRP ligand monoclonal antibodies prevent the CGRP from binding to the receptor. The receptor targeted monoclonal antibody (Erenumab) prevents internalization of the receptor in the post-synaptic neuron.

CGRP receptors are present on smooth muscle in blood vessel walls, trigeminal nerve endings particularly A delta fibers and the trigeminal ganglion. The blood brain barrier (BBB) prevents the Monoclonal antibodies from reaching central structures; but they can reach the trigeminal ganglion, trigeminal nerves, blood vessels and parts of the hypothalamus and area postrema which are outside of the BBB.

Ubrogepant, atogepant and rimegepant, on the other hand, are small molecules that may cross the BBB and non-competitively block CGRP from binding to CGRP receptors. One difference between these small molecules and the monoclonal antibodies is the target and the duration of effect on the target. Ubrogepant, atogepant and rimegepant have a much shorter half-life measured in hours compared to close to a month for the monoclonal antibodies. Due to the difference in mode of action and the ability of ubrogepant and atogepant to cross the blood brain barrier, the methods described herein provide a preferred embodiment where ubrogepant and or atogepant, or a pharmaceutically acceptable salt thereof is administered to a patient suffering from medication overuse headache.

In some embodiments, the patient is undergoing treatment for pain, and MOH is induced by the treatment for pain. In some embodiments, the patient is undergoing treatment with acute pain medications or chronic pain medications. In some embodiments, the patient is undergoing treatment with triptans, ergot alkaloids, analgesics and/or opioids. In some embodiments, the patient is undergoing treatment with a triptan selected from rizatriptan, sumatriptan, naratriptan, eletriptan, donitriptan, almotriptan, frovatriptan, avitriptan and zolmitriptan. In some embodiments, the patient is undergoing treatment with an ergot alkaloid selected from clavines, lysergic acid amides and ergopeptines. In some embodiments, the patient is undergoing treatment with an ergot alkaloid selected from ergonovine, methylergonovine, methysergide, ergotamine, dihydroergotamine, bromocriptine, ergoloid mesylates and lysergic acid diethylamide, or a combination thereof.

In some embodiments, the MOH results from the chronic use of one or more pain medications. In some embodiments, the patient has a primary headache disorder selected from migraine, cluster-type headache or tension-type headache. In some embodiments, the patient is currently undergoing treatment or has received treatment for a primary headache disorder, wherein the treatment includes a medicament selected from aspirin, diclofenac; diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib, etoricoxib, valdecoxib, parecoxib, meloxicam, lumiracoxib, or a combination thereof.

In some embodiments, the MOH results from treatment with a medicament selected from ketamine, esketamine, alfentanil, alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone and zolpidem.

In some embodiments, the MOH results from the chronic use of a medicament selected from alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone and zolpidem.

In some embodiments, the MOH results from the chronic use of a medicament selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanyl, tramadol, prochlorperazine, celecoxib, rofecoxib, meloxicam, piroxicam, JTE-522, L-745,337, NS388, deracoxib, valdecoxib, iumiracoxib, etoricoxib, parecoxib, 4-(4-cyclohexyl-2-methyloxazol-5-y1)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl) phenyl]-3(2H) pyridazinone, 2-[(2,4-dichloro-6-methylphenyl) amino]-5-ethyl-benzeneacetic acid, (3Z) 3-[(4-chlorophenyl) [4-(methyl sulfonyl)phenyl]methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, ciltiazem, Nifedipine, lidocaine, tetracaine, prilocaine, bupivicaine, mepivacaine, etidocaine, procaine, benzocaine, phehelzine, isocarboxazid, dichloralphenazone, nimopidine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth sub salicylate, buclizine, cinnarizine, cyclizine, diphenidol, dolasetron, domperidone, dronabinol, droperidol, haloperidol, metoclopramide, nabilone, thiethylperazine, trimethobenzemide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

In some embodiments, the application provides a method for treating post-traumatic headache (PTH) headache in a patient in need thereof, comprising the step of administering to said patient a therapeutically effective amount of a CGRP-antagonist. or a pharmaceutically acceptable salt thereof. In some embodiments, the patient has experienced a PTH one, two, three, four, five, six or seven days after a traumatic incident. In some embodiments, the traumatic incident resulted a concussion or loss of consciousness. In some embodiments, the patient suffers from dizziness, insomnia, poor concentration, memory problems, photophobia, phonophobia, or fatigue, or a combination thereof.

In some embodiments, the application provides a method for treating post-concussion syndrome in a patient in need thereof, comprising the step of administering to said patient a therapeutically effective amount of a CGRP-antagonist or a pharmaceutically acceptable salt thereof.

In some embodiments, the application provides a method for treating vertigo in a patient in need thereof, comprising the step of administering to said patient a therapeutically effective amount of a calcitonin gene-related peptide(CGRP-antagonist) or a pharmaceutically acceptable salt thereof.

In some embodiments, starting a CGRP antagonist in a patient with MO (medication overuse) or MOH (medication overuse headache) can help reduce headache and allow for weaning of the overused acute medication.

In some embodiments, the CGRP-antagonist is an anti-calcitonin gene-related peptide receptor antibody (anti-CGRP antibody) or antigen-binding fragment thereof. For example, the antibody can be selected from galcanezumab, fremanezumab, eptinezumab or erenumab. In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy for the treatment of migraine. CGRP-antagonists can be administered by any method that allows the antagonist to reach the intended targets. The CGRP-antagonists can be administered to the patient over the course of a set treatment period or indefinitely. The treatment can continue while the patient exhibit or experience symptoms of medication overuse headache. The treatment period can vary and in some embodiments, lasts only one day where the antagonist is administered, and in some embodiments, can continue indefinitely while the patient continues to suffer from headache. The CGRP-antagonist can be administered by many different methods including peripheral, parenteral, subcutaneous, intravenous, intraperitoneal, intracerebral, intralesional, intramuscular, intraocular, intraarterial interstitial infusion and implanted delivery device.

The anti-CGRP antibody, erenumab can be administered weekly, biweekly, monthly, every two months, every three months, every four months, every five months or every six months at a dosage of about 5 mg to about 500 mg. Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017). In one embodiment, erenumab can be administered subcutaneously at a dose of about 5 mg to about 500 mg every one, two, three, four, five, six, seven, eight, nine or ten weeks. In one embodiment, erenumab can be administered subcutaneously at a dose of about 10 mg to about 200 mg every one, two, three, four, five, six, seven, eight, nine or ten weeks. In one embodiment, erenumab can be administered subcutaneously at a dose of about 25 mg to about 150 mg every one, two, three, four, five, six, seven, eight, nine or ten weeks. In one embodiment, erenumab can be administered subcutaneously at a dose of about 90 mg to about 120 mg every one, two, three, four, five, six, seven, eight, nine or ten weeks. In one embodiment, erenumab can be administered subcutaneously at a dose of about 50 mg to about 60 mg every one, two, three, four, five, six, seven, eight, nine or ten weeks. In one embodiment, erenumab can be administered subcutaneously at a dose of about 70 mg every one, two, three, four, five, six, seven, eight, nine or ten weeks. In one embodiment, erenumab can be administered subcutaneously at a dose of about 140 mg every one, two, three, four, five, six, seven, eight, nine or ten weeks. In one embodiment, erenumab can be administered subcutaneously at a monthly dose of about 140 mg. In one embodiment, erenumab can be administered subcutaneously at a monthly dose of about 70 mg. In one embodiment, erenumab can be administered subcutaneously at a dose of about 140 mg every two months. In one embodiment, erenumab can be administered subcutaneously at a dose of about 70 mg every two months. In one embodiment, erenumab can be administered subcutaneously at a dose of about 140 mg every three months. In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg. In one embodiment, galcanezumab is administered subcutaneously at a dose of about 10 mg to about 500 mg every one, two, three, four, five, six, seven, eight, nine or ten weeks. In one embodiment, galcanezumab is administered subcutaneously at a dose of about 50 mg to about 300 mg every one, two, three, four, five, six, seven, eight, nine or ten weeks. In one embodiment, galcanezumab is administered subcutaneously at a dose of about 75 mg to about 250 mg every one, two, three, four, five, six, seven, eight, nine or ten weeks. In one embodiment, galcanezumab is administered subcutaneously at a dose of about 75 mg to about 100 mg every one, two, three, four, five, six, seven, eight, nine or ten weeks. In one embodiment, galcanezumab is administered subcutaneously at a dose of about 150 mg to about 220 mg every one, two, three, four, five, six, seven, eight, nine or ten weeks. In one embodiment, galcanezumab is administered subcutaneously at a dose of about 120 mg every one, two, three, four, five, six, seven, eight, nine or ten weeks. In one embodiment, galcanezumab is administered subcutaneously at a dose of about 240 mg every one, two, three, four, five, six, seven, eight, nine or ten weeks. In one embodiment, galcanezumab is administered subcutaneously at a monthly dose of about 240 mg. In one embodiment, galcanezumab is administered subcutaneously at a monthly dose of about 120 mg. In one embodiment, galcanezumab is administered subcutaneously at a dose of about 240 mg every two months. In one embodiment, galcanezumab is administered subcutaneously at a dose of about 120 mg every two months. In one embodiment, galcanezumab is administered subcutaneously at a dose of about 240 mg every three months. In one embodiment, galcanezumab is administered subcutaneously at a dose of about 120 mg every three months.

In one embodiment, fremanezumab is administered subcutaneously at a dose of about 100 mg to about 1000 mg every one, two, three, four, five, six, seven, eight, nine or ten weeks. In one embodiment, fremanezumab is administered subcutaneously at a dose of about 150 mg to about 700 mg every one, two, three, four, five, six, seven, eight, nine or ten weeks. In one embodiment, fremanezumab is administered subcutaneously at a dose of about 150 mg to about 500 mg every one, two, three, four, five, six, seven, eight, nine or ten weeks. In one embodiment, fremanezumab is administered subcutaneously at a dose of about 150 mg to about 200 mg every one, two, three, four, five, six, seven, eight, nine or ten weeks. In one embodiment, fremanezumab is administered subcutaneously at a dose of about 150 mg to about 500 mg every one, two, three, four, five, six, seven, eight, nine or ten weeks. In one embodiment, fremanezumab is administered subcutaneously at a dose of about 225 mg every one, two, three, four, five, six, seven, eight, nine or ten weeks. In one embodiment, fremanezumab is administered subcutaneously at a dose of about 450 mg every one, two, three, four, five, six, seven, eight, nine or ten weeks. In one embodiment, fremanezumab is administered subcutaneously at a dose of about 675 mg every one, two, three, four, five, six, seven, eight, nine or ten weeks. In one embodiment, fremanezumab is administered subcutaneously at a monthly dose of about 225 mg. In one embodiment, fremanezumab is administered subcutaneously at a monthly dose of about 450 mg. In one embodiment, fremanezumab is administered subcutaneously at a monthly dose of about 675 mg. In one embodiment, fremanezumab is administered subcutaneously at a dose of about 225 mg every two months. In one embodiment, fremanezumab is administered subcutaneously at a dose of about 450 mg every two months. In one embodiment, fremanezumab is administered subcutaneously at a dose of about 225 mg every three months. In one embodiment, fremanezumab is administered subcutaneously at a dose of about 450 mg every three months. In one embodiment, fremanezumab is administered subcutaneously at a dose of about 675 mg every three months.

In one embodiment, eptinezumab is administered subcutaneously at a dose of about 50 mg to about 1000 mg every one, two, three, four, five, six, seven, eight, nine or ten weeks. In one embodiment, eptinezumab is administered subcutaneously at a dose of about 100 mg to about 700 mg every one, two, three, four, five, six, seven, eight, nine or ten weeks. In one embodiment, eptinezumab is administered subcutaneously at a dose of about 200 mg to about 500 mg every one, two, three, four, five, six, seven, eight, nine or ten weeks. In one embodiment, eptinezumab is administered subcutaneously at a dose of about 250 mg to about 350 mg every one, two, three, four, five, six, seven, eight, nine or ten weeks. In one embodiment, eptinezumab is administered subcutaneously at a dose of about 300 mg every one, two, three, four, five, six, seven, eight, nine or ten weeks. In one embodiment, eptinezumab is administered subcutaneously at a monthly dose of about 100 mg. In one embodiment, eptinezumab is administered subcutaneously at a monthly dose of about 200 mg. In one embodiment, eptinezumab is administered subcutaneously at a monthly dose of about 300 mg. In one embodiment, eptinezumab is administered subcutaneously at a dose of about 100 mg every two months. In one embodiment, eptinezumab is administered subcutaneously at a dose of about 200 mg every two months. In one embodiment, eptinezumab is administered subcutaneously at a dose of about 300 mg every two months. In one embodiment, eptinezumab is administered subcutaneously at a dose of about 100 mg every three months. In one embodiment, eptinezumab is administered subcutaneously at a dose of about 200 mg every three months. In one embodiment, eptinezumab is administered subcutaneously at a dose of about 300 mg every three months.

Preferably, the CGRP antagonist is selected from ubrogepant, atogepant, rimegepant or a pharmaceutically acceptable salt thereof.

In some embodiments, the CGRP antagonist is ubrogepant. In some embodiments, ubrogepant is administered at an oral dose of about 5 to about 500 mg once, twice or three times a day. In some embodiments, ubrogepant is administered at an oral dose of about 25 mg once, twice or three times a day. In some embodiments, ubrogepant is administered at an oral dose of about 50 mg once, twice or three times a day. In some embodiments, ubrogepant is administered at an oral dose of about 100 mg once, twice or three times a day. In some embodiments, ubrogepant is administered at an oral dose of about 200 mg once, twice or three times a day.

In some embodiments the CGRP antagonist is atogepant. In some embodiments, atogepant is administered at an oral dose of about 5 to about 500 mg once, twice or three times a day. In some embodiments, atogepant is administered at an oral dose of about 25 mg once, twice or three times a day. In some embodiments, atogepant is administered at an oral dose of about 50 mg once, twice or three times a day. In some embodiments, atogepant is administered at an oral dose of about 100 mg once, twice or three times a day. In some embodiments, atogepant is administered at an oral dose of about 200 mg once, twice or three times a day.

In some embodiments, the CGRP antagonist is rimegepant. In some embodiments, rimegepant is administered at an oral dose of about 5 to about 500 mg once, twice or three times a day. In some embodiments, rimegepant is administered at an oral dose of about 25 mg once, twice or three times a day. In some embodiments, rimegepant is administered at an oral dose of about 50 mg once, twice or three times a day. In some embodiments, rimegepant is administered at an oral dose of about 100 mg once, twice or three times a day. In some embodiments, rimegepant is administered at an oral dose of about 200 mg once, twice or three times a day. In some embodiments, rimegepant is administered at an oral dose of about 5 to about 500 mg once, twice or three times a day. In some embodiments, rimegepant is administered at an oral dose of about 25 mg once, twice or three times a day. In some embodiments, rimegepant is administered at an oral dose of about 50 mg once, twice or three times a day. In some embodiments, rimegepant is administered at an oral dose of about 100 mg once, twice or three times a day. In some embodiments, rimegepant is administered at an oral dose of about 200 mg once, twice or three times a day.

In some embodiments, the CGRP-antagonist can be administered orally, sublingually, transdermally, subcutaneously, intravenously, or intramuscularly.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Effective amount” as applied to the biologically active ingredient means that amount of the ingredient which is generally sufficient to effect a desired change in the subject. For example, where the desired effect is a reduction in an autoimmune disorder symptom, an effective amount of the ingredient is that amount which causes at least a substantial reduction of the autoimmune disorder symptom, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra—or subdermal injection or topical administration. Local administration excludes systemic routes of administration, such as intravenous or oral administration. Topical administration is a type of local administration in which a pharmaceutical agent is applied to a patient's skin.

“Patient” means a human or non-human subject receiving medical or veterinary care. Accordingly, the compositions as disclosed herein can be used in treating any animal, such as, for example, mammals, or the like.

“Peripheral administration” means administration by means of a non-systemic route to a peripheral location on a mammal. Peripheral administration includes subdermal, intranasal, intramuscular, intradermal, transdermal, and subcutaneous administration.

“Pharmaceutical composition” means a composition comprising an active pharmaceutical ingredient, such as, for example, a CGRP antagonist, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

“Tonicity agent” means a low molecular weight excipient which is included in a formulation to provide isotonicity. Disaccharide, such as trehalose or sucrose, polyalcohol, such as sorbitol or mannitol, monosaccharide, such as glucose, and salt, such as sodium chloride, can serve as a tonicity agent.

“Polysaccharide” means a polymer of more than two saccharide molecule monomers. The monomers can be identical or different.

“Stabilizers” can include excipients, and can include protein and non-protein molecules.

“Therapeutic formulation” means a formulation can be used to treat and thereby alleviate a disorder or a disease, such as, for example, a disorder or a disease characterized by hyperactivity (i.e. spasticity) of a peripheral muscle.

“Treating” means to alleviate (or to eliminate) at least one symptom of a condition or disorder, such as, for example, wrinkles, spasticity, depression, pain (such as, for example, headache pain), bladder overactivity, or the like, either temporarily or permanently.

### EXAMPLES

The following non-limiting examples provide those of ordinary skill in the art with possible case scenarios and specific methods to treat conditions within the scope of the present disclosure and are not intended to limit the scope of the disclosure.

### Example 1

A 38-year-old woman complains of right-sided frontal headaches. The patient stated the headaches began 3 months ago after accidentally being hit in the face at the right frontal, maxillary, and zygomatic bones with a heavy instrument. She denies any loss of consciousness at the time of the incident and describes the pain as a “constant achy” to “stabbing” pain primarily over the right eye and right zygomatic bone. She complains of intermittent blurred vision when the pain becomes “stabbing.” She also complains of intermittent tinnitus. On a subjective rating scale of zero (no pain) to 10 (worst pain), the patient rates her pain as eight at best, nine on average, and 10 at worst. Results from a radiographic scan of the skull, CT scan of the brain, and MRI scan of the brain and cervical spine are normal. Results from slit lamp and neurologic testing are also normal. The patient is diagnosed to have PTH. The patient can be treated with ubrogepant at a dose of 100 mg, once a day. The treatment with ubrogepant can reduce the pain experienced by the patient and the patient can rate her pain as five at best, six on average, and nine at worst.

### Example 2

A 45-year-old woman have been experiencing migraine attacks since her early teens which was not associated with any visual, sensory or language symptoms. This responded well to Excedrin at that time. As she became older her headaches became more frequent and by the time she was in her late 20's she had headache on more than half the days of the month. Excedrin was no longer effective, and her primary care physician prescribed Fiorinal (aspirin, caffeine and barbiturate compound). By her early 40's she has been experiencing daily headache and used Fiorinal four times a day: on waking and every 6 hours throughout the day and night, and she would often awaken in the early morning with a severe headache. These headaches could be associated with nausea, photophobia and phonophobia and often had a throbbing quality. Her primary care physician, on routine blood work noted: worsening renal and liver function tests. The patient reported depression and disability related to her headache disorder. She could not work or participate in family events. She was referred to a neurologist who specialized in headache disorders. He diagnosed her with episodic migraine without aura that had transformed to chronic migraine with associated medication overuse headache. He also diagnosed end organ damage with hepatic and renal damage secondary to analgesic overuse. He recommended a preventive treatment and that she taper off of Fiorinal. The patient started Topiramate as a migraine preventive and despite her best efforts she was unable to wean off of Fiorinal, due to breakthrough headache. She returned to the Neurologist and he prescribed Ubrogepant 50 mg daily for 10 days. During this time, she was able to successfully wean off of Fiorinal. She continued Topiramate treatment and used Ubrogepant 50 mg a day as needed for breakthrough headache. She found that in the first month after she was weaned off of Fiorinal, she only needed to use this twice a week and after 3 months she was only needing to use Ubrogepant once or twice a month. This effectively ended her migraine attacks and she had no recurrence requiring re-treatment. She was able to return to work and to participate in family events.

### Example 3

A 52-year-old woman visits a headache clinic with almost daily migraine attacks. Her migraines have been ongoing since she was 25 years of age. She treats her migraine attacks with an oral triptan, which initially was highly effective. Eventually, however, the migraine attacks became longer, lasting up to 3 or 4 days and forcing her to take 4 or 5 doses of triptan per attack. Currently, she is averaging 25 doses of oral triptan per month. She is diagnosed with migraine without aura and medication overuse headache. She is treated with ubrogepant at a dose of 50 mg, once a day. The treatment with ubrogepant can reduce the intensity and frequency of migraine experienced by the patient.

### Example 4

A 38-year-old woman complains of right-sided frontal headaches. The patient stated the headaches began 3 months ago after accidentally being hit in the face at the right frontal, maxillary, and zygomatic bones with a heavy instrument. She denies any loss of consciousness at the time of the incident and describes the pain as a “constant achy” to “stabbing” pain primarily over the right eye and right zygomatic bone. She complains of intermittent blurred vision when the pain becomes “stabbing.” She also complains of intermittent tinnitus. On a subjective rating scale of zero (no pain) to 10 (worst pain), the patient rates her pain as eight at best, nine on average, and 10 at worst. Results from a radiographic scan of the skull, CT scan of the brain, and MRI scan of the brain and cervical spine are normal. Results from slit lamp and neurologic testing are also normal. The patient is diagnosed to have PTH. The patient can be treated with erenumab at a dose of 70 mg by subcutaneous administration. A month after the erenumab administration, the patient can rate her pain as five at best, six on average, and nine at worst.

1. A method for treating medication-overuse headache (MOH) in a patient in need thereof, comprising the step of administering to said patient a therapeutically effective amount of a calcitonin gene-related peptide antagonist (CGRP-antagonist) or a pharmaceutically acceptable salt thereof.

2. The method according to claim 1 wherein said patient is undergoing treatment for pain, wherein said treatment for pain includes a medicament selected from the group consisting of acute pain medications and chronic pain medications.

3. (canceled)

4. (canceled)

5. The method according to claim 2, wherein said medicament is selected from the group consisting of:
a triptan selected from the group consisting of rizatriptan, sumatriptan, naratriptan, eletriptan, donitriptan, almotriptan, frovatriptan, avitriptan and zolmitriptan;
an ergot alkaloid selected from the group consisting of clavines, lysergic acid amides, ergopeptines, ergovine, methylergonovine, methysergide, ergotamine, dihydroergotamine, bromocriptine, ergoloid mesylates and lysergic acid diethylamide, or a combination thereof; and
a medicament selected from the group consisting of aspirin, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib, etoricoxib, valdecoxib, parecoxib, meloxicam, lumiracoxib, or a combination thereof.

6-8. (canceled)

9. The method according to claim 1 wherein said patient has a primary headache disorder selected from migraine, cluster-type headache or tension-type headache, and wherein said patient is currently undergoing treatment or has received treatment for said primary headache disorder.

10. (canceled)

11. (canceled)

12. The method according to any of claim 1 wherein said MOH results from treatment or use of a medicament selected from the group consisting of:
treatment with a medicament selected from ketamine, esketamine, alfentanil, alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone and zolpidem;
chronic use of a medicament selected from alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone and zolpidem; and
chronic use of a medicament selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanyl, tramadol, prochlorperazine, celecoxib, rofecoxib, meloxicam, piroxicam, JTE-522, L-745,337, NS388, deracoxib, valdecoxib, iumiracoxib, etoricoxib, parecoxib, 4-(4-cyclohexyl-2-methyloxazol-5-y1)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl) phenyl]-3(2H) pyridazinone, 2-[(2,4-dichloro-6-methylphenyl)amino]-5-ethyl-benzeneacetic acid, (3Z) 3-[(4-chlorophenyl) [4-(methyl sulfonyl)phenyl] methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, ciltiazem, Nifedipine, lidocaine, tetracaine, prilocaine, bupivicaine, mepivacaine, etidocaine, procaine, benzocaine, phehelzine, isocarboxazid, dichloralphenazone, nimopidine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth sub salicylate, buclizine, cinnarizine, cyclizine, diphenidol, dolasetron, domperidone, dronabinol, droperidol, haloperidol, metoclopramide, nabilone, thiethylperazine, trimethobenzemide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

13. (canceled)

14. (canceled)

15. A method for treating a condition selected from the group consisting of post-traumatic headache (PTH), post-concussion syndrome, and vertigo in a patient in need thereof, comprising the step of administering to said patient a therapeutically effective amount of a calcitonin gene-related peptide antagonist (CGRP-antagonist) or a pharmaceutically acceptable salt thereof.

16-20. (canceled)

21. The method according to claim 1 wherein said CGRP-antagonist is an anti-calcitonin gene-related peptide receptor antibody (anti-CGRP antibody).

22. The method according to claim 21 wherein said antibody is selected from the group consisting of galcanezumab, fremanezumab, eptinezumab, and erenumab.

23. The method claim 1 wherein said CGRP-antagonist is selected from the group consisting of ubrogepant, atogepant, rimegepant or a pharmaceutically acceptable salt thereof.

24. The method according to claim 22 wherein said anti-CGRP antibody is erenumab.

25. The method according to claim 24 wherein erenumab is administered subcutaneously at a dose of about 5 mg to about 500 mg every one, two, three, four, five, six, seven, eight, nine or ten weeks.

26-37. (canceled)

38. The method according to claim 22 wherein said anti-CGRP antibody is galcanezumab.

39. The method according to claim 38 wherein galcanezumab is administered subcutaneously at a dose of about 10 mg to about 500 mg every one, two, three, four, five, six, seven, eight, nine or ten weeks.

40-51. (canceled)

52. The method according to claim 22 wherein said anti-CGRP antibody is fremanezumab.

53. The method according to claim 52 wherein fremanezumab is administered subcutaneously at a dose of about 100 mg to about 1000 mg every one, two, three, four, five, six, seven, eight, nine or ten weeks.

54-68. (canceled)

69. The method according to claim 22 wherein said anti-CGRP antibody is eptinezumab.

70. The method according to claim 69 wherein eptinezumab is administered subcutaneously at a dose of about 50 mg to about 1000 mg every one, two, three, four, five, six, seven, eight, nine or ten weeks.

71-83. (canceled)

84. The method according to claim 23 wherein said CGRP-antagonist is ubrogepant, and said ubrogepant is administered at an oral dose of about 5 to about 500 mg once, twice or three times a day.

85-88. (canceled)

89. The method according to claim 23 wherein said CGRP-antagonist is atogepant, and said atogepant is administered at an oral dose of about 5 to about 500 mg once, twice or three times a day.

90-93. (canceled)

94. The method according to claim 23 wherein said CGRP-antagonist is rimegepant, and said rimegepant is administered at an oral dose of about 5 to about 500 mg once, twice or three times a day.

95-103. (canceled)
